(19) United States
(12) Patent Application Publication
(10) Pub. No.: US 2022/0226365 A1
(43) Pub. Date: Jul. 21, 2022

(54) LIGHT-CURING COMPOSITIONS FOR TREATING ONYCHOMYCOSIS (FUNGAL NAIL INFECTION)

(71) Applicant: L/N HEALTH AND BEAUTY APS, Lystrup (DK)

(72) Inventor: Diana Hoejberg, Risskov (DK)

(21) Appl. No.: 17/611,999

(22) PCT Filed: May 15, 2020
(86) PCT No.: PCT/IB2020/054610

§ 371 Date: Nov. 17, 2021

(30) Foreign Application Priority Data
May 17, 2019 (DE) 10 2019 003 486.4
Dec. 18, 2019 (DE) 10 2019 008 797.6

(51) Int. Cl. A61K 31/78; A61P 31/10; C08F 2/50

(52) U.S. Cl. CPC A61K 31/78; C08F 2/50; A61P 31/10

(57) ABSTRACT

The present invention relates to photopolymerizable acrylate-based compositions for producing a light-curing lacquer for the therapy of onychomycosis or of a bacterial nail infection for a human or animal toenail or fingernail, the compositions being devoid of a classic antimycotic active ingredient. Various different acrylates can be used to produce the claimed compositions.

### BACKGROUND OF THE INVENTION

The present invention relates to compositions for treating onychomycosis being devoid of a classic antimycotic active ingredient.

Onychomycosis (bacterial nail infection) is not a serious, but a frequent disease. According to surveys, 5-12% of Europeans have dermatophytes in the nails, and the frequency increases with age.

The classic treatment is, in a simple case, by an antimycotic cream or an antimycotic nail polish. The nail polish contains an antimycotic agent, such as bifonazole, clotrimazole, ciclopirox, or amorolfine. For serious diseases, surgical removal of the nail may be necessary, there is, further, frequently a necessity of systemic administration of oral antimycotic agents such as griseofulvin, itraconazole, terbinafine, or fluconazole, in turn, presenting risks and side effects.

There is, therefore, a further demand for efficient treatment options for onychomycoses.

WO 2018/207164 A1 describes a kit for the correction of improperly grown toenails or fingernails, by means of which a photopolymerizable substance is applied onto the nail, in order to mechanically correct it. The document also mentions an optional embodiment with addition of antimycotic agents, in order to also perform, simultaneously with the mechanical correction, an antimycotic therapy.

### SUMMARY OF THE INVENTION

The present invention relates to the use of a photopolymerizable acrylate-based composition for producing a light-curing lacquer for the therapy of onychomycosis or of a bacterial nail infection.

Photopolymerizable acrylate-based compositions according to the invention for producing a light-curing lacquer for the therapy of onychomycosis or of a bacterial nail infection, in particular, comprise an aliphatic urethane dimethacrylate, hydroxyethylene methacrylate, phosphate dimethacrylate, triethylene glycol dimethacrylate, bis-phenyl glycidyl dimethacrylate, triethylene glycol dimethacrylate, alkoxylated bisphenol-A dimethacrylate, isopropylidene diphenyl-bis-oxyhydroxy propyl methacrylate, 2-hydroxyethyl methacrylate, polyester polyol tetra-acrylate, or mixtures of the above-mentioned components in combination with a starter.

The present invention relates, for instance, to the use of a composition,

comprising

15-45% of bisphenol-(A) dimethacrylate, urethane dimethacrylate in a ratio of 1:5 to 5:1,

85-55% of additives

0.1-1% of camphorquinone, amino starter,

for producing a light-curing lacquer for the therapy of onychomycosis or of a bacterial nail infection.

The material for the light-curing lacquer includes additives, in particular fillers and pigments.

These are, first, silicate-based fillers (silica fillers), fillers based on ground barium glass (barium glass fillers), and polymer particles. The particulate additives have diameters in the range from 0.1-10 μm, preferably, they are smaller than 5 μm. In particular, the fillers based on barium glass considerably contribute to the mechanical properties of the light-curing lacquer. In addition, pigments may be included in the composition, in order to give the formed lacquer an esthetic appearance. The pigments, too, should preferably have diameters in the range from 0.1-10 μm.

Further potential additives of the compositions are:

a) solvents, such as ethanol, propanol, ethyl acetate,

b) film-forming substances, such as, e.g., Di-HEMA trimethylhexyl dicarbamate,

c) antioxidants, such as butylated hydroxytoluene (BHT), butylated hydroxyanisole (BHA), or pentaerythrityl tetra-di-t-butyl hydroxycinnamate,

d) odorants.

The compositions according to the invention further comprise polymerization starters that can initiate the desired polymerization reaction by irradiation with light. For this purpose, in principle, all classic polymerization starters are suitable. Particularly suitable is the combination of camphorquinone with amino starters, namely tertiary amines (e.g., triethanolamine, N,N-dimethyl-p-toluidine, triethylamine, 4-dimethylamino benzoic acid ethyl ester, N,N-tetramethylaniline). Alternatively, for instance, 2-ethylanthraquinone in combination with N-phenylglycine or acylphosphine can be used.

Surprisingly, it has been found that the compositions described in WO 2018/207164 A1 are particularly well suited for the therapy of onychomycosis, even when the compositions do not contain an antimycotic agent.

The kit described in WO 2018/207164 A1 for nail correction comprises

a) primers, comprising

40-60% of hydroxyethylene methacrylate,

40-60% of phosphate dimethacrylate,

0.1-1.0% of a starter,

b) at least one composition for producing a light-curing nail brace, comprising

15-45% of bisphenol-(A) dimethacrylate, urethane dimethacrylate in a ratio of 1:5 to 5:1,

85-55% of fillers and pigments,

0.1-1% of camphorquinone, amino starter.

The component a of the kit according to the invention is a primer that acts as an adhesion promoter for the application described in WO 2018/207104 A1. In the context of the investigations regarding the present invention, it has been found that the primer is responsible, most likely, for the antimycotic effect of the composition.

The adhesion promoter comprises 40-60% of hydroxyethylene methacrylate and 40-60% of phosphate dimethacrylate as well as 0.1-1.0% of polymerization starters. The polymerization starters are described below in more detail. The mixing ratio can vary within the above-mentioned percentages. Advantageously, the two methacrylates are present in an approximately identical ratio. It is understood that all components together result in 100%.

The nail brace further described in WO 2018/207164 A1 is formed by means of the photopolymerizable material b. This is a composition comprising

15-45% of bisphenol-(A) dimethacrylate, urethane dimethacrylate in a ratio of 1:5 to 5:1,

85-55% of fillers and pigments
0.1-1% of camphorquinone, amino starter.

The above-mentioned ranges of the compositions permit a varying adjustment of the mechanical properties in the form of different hardnesses. In the practice, it has proven to provide two compositions, one of which is relatively soft, and the other one is relatively hard.

The soft composition comprises, for instance,

17-21% of bisphenol-(A) dimethacrylate, urethane dimethacrylate in a ratio of 1:4 to 4:1,

79-83% of fillers and pigments,

0.1-1% of camphorquinone, amino starter.

The hard composition comprises, for instance,

36-40% of bisphenol-(A) dimethacrylate, urethane dimethacrylate in a ratio of 1:4 to 4:1,

60-64% of fillers and pigments,

0.1-1% of camphorquinone, amino starter.

The monomers bisphenol-(A) dimethacrylate and urethane dimethacrylate included in the compositions are preferably included in an approximately identical amount. Mixing ratios, wherein the two main components are included in a mass ratio between 1:5 and 5:1, are generally suitable. Particularly preferred are mixing ratios, wherein two main components are included in a mass ratio between 1:2 and 2:1.

The material for the light-curing nail brace further comprises fillers and pigments. These are silicate-based fillers (silica fillers}, fillers based on ground barium glass (barium glass fillers) as well as polymer particles. The particulate additives have diameters in the range from 0.1-10 μm, preferably they are smaller than 5 μm. In particular, the fillers based on barium glass considerably contribute to the mechanical properties of the nail brace. In addition, pigments may be included in the composition, in order to give the formed nail brace an esthetic appearance. The pigments, too, should preferably have diameters in the range from 0.1-10 μm.

Further potential components of the compositions are:

a) solvents, such as ethanol, propanol, ethyl acetate,

b) film-forming substances, such as, e.g., Di-HEMA trimethylhexyl dicarbamate,

c) antioxidants, such as butylated hydroxytoluene (BHT), butylated hydroxyanisole (BHA), or pentaerythrityl tetra-di-t-butyl hydroxycinnamate,

d) odorants.

Obviously, the material of the light-curing nail brace according to WO 2018/207164 A1 contributes to the success of the therapy for onychomycosis, but it seems not to be indispensable. A therapy of onychomycosis by application of the material of the light-curing nail brace alone appears to be possible, while a therapy of onychomycosis by application of the light-curing primer alone is preferred. An application of both components (primer and material of the light-curing brace) is particularly preferred.

The compositions according to the invention further comprise polymerization starters that can initiate the desired polymerization reaction by irradiation with light. For this purpose, in principle, all classic polymerization starters are suitable. Particularly suitable is the combination of camphorquinone with amino starters, namely tertiary amines (e.g., triethanolamine, N,N-dimethyl-p-toluidine, triethylamine, 4-dimethylamino benzoic acid ethyl ester, N,N-tetramethylaniline). Alternatively, for instance, 2-ethylanthraquinone in combination with N-phenylglycine or acylphosphine can be used.

### BRIEF DESCRIPTION OF THE DRAWINGS

FIG. 1 shows a human toenail before the therapy.

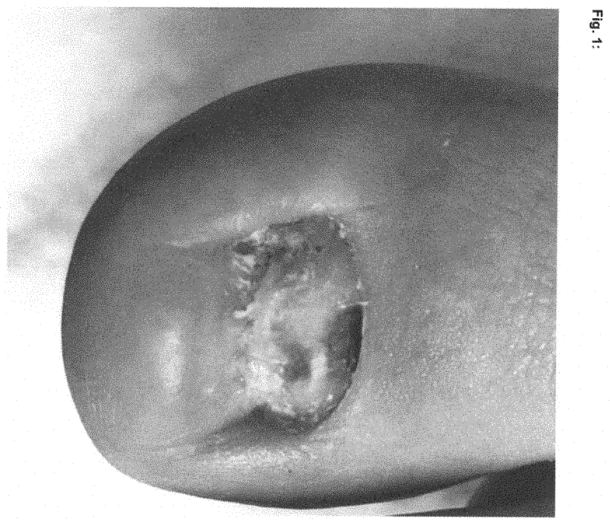

FIG. 2 shows the same toenail after the therapy.

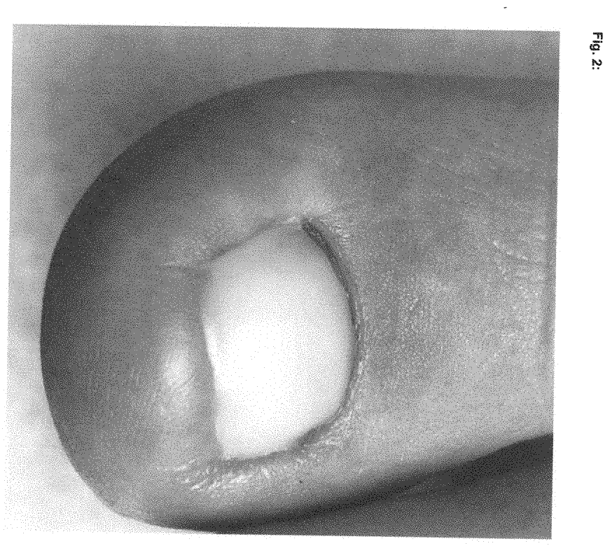

### DETAILED DESCRIPTION OF THE INVENTION

The present invention relates, therefore, in particular to the use of a composition,

comprising

40-60% of hydroxyethylene methacrylate

40-60% of phosphate dimethacrylate,

0.1-1.0% of a starter,

for producing a light-curing lacquer for the therapy of onychomycosis or of a bacterial nail infection.

The present invention further relates to the use of a composition,

comprising

15-45% of bisphenol-(A) dimethacrylate, urethane dimethacrylate in a ratio of 1:5 to 5:1,

85-55% of fillers and pigments

0.1-1% of camphorquinone, amino starter,

for producing a light-curing lacquer for the therapy of onychomycosis or of a bacterial nail infection.

Further embodiments of the invention are subject matter of the claims.

When using such a system, the completed composition may surprisingly be stored over a longer time, without a polymerization reaction taking place. Only after irradiation with a suitable light source, the polymerization reaction will take place. For this purpose, e.g., an LED light source with a wavelength of approx. 425 nm and a light power of 1000-1500 mW/cm² can be used.

The invention can be used for a nail correction treatment, as described in WO 2018/207164 A1. In addition to the mechanical correction of the nail, a potentially existing onychomycosis is also treated in parallel.

The kit according to the invention may, however, also be used without a nail correction treatment. For this purpose, first, the primer is applied on the surface of the respective nail. Care has, in particular, to be taken that the nail is dry. The respective nail should, in particular in the 24 hours before the application, not have been in contact with water. The patient should, for instance, not have taken a bath. Short washing or showering is, however, harmless, if the nail has been thoroughly dried. If necessary, the nail may be dried with a hot-air blower. After application, the polymerization is started by means of a light source (preferably blue light with approx. 425 nm and at least 1000 mW/cm²). When using a usual light source, the polymerization is completed after a period of time of 5 seconds to 60 seconds, normally a 10-second irradiation is sufficient. Then, the light-curing nail brace is applied. In the case of a pure antimycotic treatment, the application may be effected on the surface. If simultaneously a mechanical correction is to be performed, the application takes place as described in WO 2018/207164 A1. After the application, the polymerization occurs preferably immediately by irradiation with the above-mentioned light source. It is important, when doing so, to hold the nail in the desired shape. This step, too, is usually completed after a period of time of 5-60 seconds. Then, the material may again be reground, so that no sharp edges are formed, where tissue (e.g., stockings) may be caught.

As already described above, in many cases, the application of the primer is sufficient for the therapy of onychomycosis.

The compositions according to the invention are preferably offered in correspondingly designed containers. For the primer, in principle, glass or plastic vials with an application brush are suitable. The compositions for producing light-curing nail braces are typically more viscous and are preferably offered in cartridges for use together with a cartridge press or pistol. Preferably, all containers are preferably optically opaque.

With the compositions according to the invention, the necessary material for the effective treatment of onychomycoses is provided, without classic antimycotic agents being required locally or systemically. Surprisingly, it has been found that the described compositions are also suitable to effectively fight bacterial infections of the nails (e.g., by Staphylococcus aureus, streptococci, or Pseudomonas aeruginosa). The application of the compositions on the respective nail is performed as described above for onychomycosis. Optionally, the compositions according to the invention may also include antimycotically and/or antibacterially acting therapeutic agents, such as piroctone olamine.

### EXAMPLES

The invention will be explained in more detail by the following exemplary compositions:
A) Primer

| | A1 | A2 | A3 | A4 |
|---|---|---|---|---|
| Component | (wt. %) | (wt. %) | (wt. %) | (wt. %) |
| Hydroxyethylene methacrylate | 49.7 | 39.7 | 35.7 | 45.7 |
| Phosphate dimethacrylate | 49.7 | 59.7 | 54.6 | 44.7 |
| [Bis(glyceryl | | | | |
| dimeth-acrylate) | | | | |
| phosphate] | | | | |
| Camphorquinone | 0.4 | 0.4 | 0.5 | 0.4 |
| Triethylamine | 0.2 | | 0.1 | 0.2 |
| N,N-Dimethyl-p-toluidine | | 0.2 | 0.1 | |

B) Nail brace (soft)

| | B1 | B2 | B3 | B4 | B5 |
|---|---|---|---|---|---|
| Component | (wt. %) | (wt. %) | (wt. %) | (wt. %) | (wt. %) |
| Bisphenol-(A) | 16.0 | 15.0 | 14.0 | 30.0 | 22.0 |
| dimethacrylate | | | | | |
| Urethane | 16.0 | 30.0 | 30.0 | 15.0 | 22.0 |
| dimethacrylate | | | | | |
| Silica filler | 20.0 | 0.0 | 17.0 | 16.5 | 18.5 |
| (Aerosil 9200) | | | | | |
| Silica filler | 5.0 | 10.3 | 15.0 | 12.5 | 4.5 |
| (Aerosil 7200) | | | | | |
| Barium glass | 18.0 | 13.0 | 13.0 | 12.0 | 14.5.0 |
| (median particle | | | | | |
| size: 13 μm) | | | | | |
| Barium glass | 3.9 | 14.0 | 5.2 | 5.0 | 5.5 |
| (median particle | | | | | |
| size: 5 μm) | | | | | |
| Polymer particles | 20.0 | 12.0 | 4.0 | 4.5 | 8.0 |
| (median particle | | | | | |
| size: 10 μm) | | | | | |
| Polymer particles | 0.0 | 5.0 | 1.0 | 3.5 | 4.0 |
| (median particle | | | | | |
| size: 6 μm) | | | | | |
| Camphorquinone | 0.6 | 0.4 | 0.5 | 0.6 | 0.0 |
| Triethylamine | 0.5 | 0.0 | 0.1 | 0.4 | 0.0 |
| N.N-Dimethyl-p- | 0.0 | 0.3 | 0.2 | 0.0 | 0.0 |
| toluidine | | | | | |
| 2-Ethylanthraquinone | 0.0 | 0.0 | 0.0 | 0.0 | 0.6 |
| N-phenylglycine | 0.0 | 0.0 | 0.0 | 0.0 | 0.4 |

C) Nail brace (hard)

| | C1 | C2 | C3 | C4 | C5 |
|---|---|---|---|---|---|
| Component | (wt. %) | (wt. %) | (wt. %) | (wt. %) | (wt. %) |
| Bisphenol-(A) | 20.0 | 25.0 | 18.0 | 19.0 | 15.0 |
| dimethacrylate | | | | | |
| Urethane | 20.0 | 13.0 | 20.0 | 20.0 | 23.0 |
| dimethacrylate | | | | | |
| Silica filler | 20.0 | 0.0 | 18.0 | 16.5 | 19.5 |
| (Aerosil 9200) | | | | | |
| Silica filler | 5.0 | 13.3 | 16.0 | 15.5 | 5.5 |
| (Aerosil 7200) | | | | | |
| Barium glass | 16.0 | 14.0 | 12.5 | 12.0 | 12.5 |
| (median particle | | | | | |
| size: 13 μm) | | | | | |
| Barium glass | 3.9 | 16.1 | 7.0 | 6.5 | 8.5 |
| (median particle | | | | | |
| size: 5 μm) | | | | | |
| Polymer | 14.0 | 12.0 | 6.2 | 6.0 | 11.0 |
| particles | | | | | |
| (median particle | | | | | |
| size: 10 μm) | | | | | |
| Polymer | 0.0 | 5.0 | 1.0 | 3.5 | 4.0 |
| particles | | | | | |
| (median particle | | | | | |
| size: 6 μm) | | | | | |
| Camphorquinone | 0.6 | 0.8 | 0.7 | 0.6 | 0.0 |
| Triethylamine | 0.5 | 0.0 | 0.2 | 0.4 | 0.0 |
| N,N-Dimethyl-p- | 0.0 | 0.8 | 0.4 | 0.0 | 0.0 |
| toluidine | | | | | |
| 2-Ethylanthraquinone | 0.0 | 0.0 | 0.0 | 0.0 | 0.6 |
| N-Phenylglycine | 0.0 | 0.0 | 0.0 | 0.0 | 0.4 |

1. Use of a photopolymerizable acrylate-based composition for producing a light-curing lacquer for the therapy of onychomycosis or of a bacterial nail infection.

2. The use of a composition according to claim 1, comprising
aliphatic urethane dimethacrylate,
hydroxyethylene methacrylate,
phosphate dimethacrylate,
triethylene glycol dimethacrylate,
bis-phenyl glycidyl dimethacrylate,
triethylene glycol dimethacrylate,
alkoxylated bisphenol-A dimethacrylate,
isopropylidene diphenyl-bis-oxyhydroxy propyl methacrylate,
2-hydroxyethyl methacrylate,
polyester polyol tetra-acrylate,
or mixtures of the above-mentioned components in combination with a starter, for producing a light-curing lacquer for the therapy of onychomycosis or of a bacterial nail infection.

3. The use of a composition according to claim 12, comprising
40-60% of hydroxyethylene methacrylate
40-60% of phosphate dimethacrylate,
0.1-1.0% of a starter,
for producing a light-curing lacquer for the therapy onychomycosis or of a bacterial nail infection.

4. The use of a composition according to claim 1, comprising
15-45% of bisphenol-(A) dimethacrylate, urethane dimethacrylate in a ratio of 1:5 to 5:1,
85-55% of fillers and pigments
0.1-1% of camphorquinone, amino starter,
for producing a light-curing lacquer for the therapy of onychomycosis or of a bacterial nail infection.

5. A kit for treating onychomycoses or a bacterial nail infection, comprising two compositions, namely a first composition comprising
40-60% of hydroxyethylene methacrylate
40-60% of phosphate dimethacrylate,
0.1-1.0% of a starter,
and a second composition comprising
15-45% of bisphenol-(A) dimethacrylate, urethane dimethacrylate in a ratio of 1:5 to 5:1,
85-55% of fillers and pigments
0.1-1% of camphorquinone, amino starter,
for producing a light-curing two-layer lacquer for the therapy of onychomycosis or of a bacterial nail infection.

6. The kit for treating onychomycoses or a bacterial nail infection according to claim 4, wherein the mass ratio of bisphenol (A) dimethacrylate to urethane dimethacrylate in the compositions for producing light-curing nail braces is in the range from 1:2 to 2:1, preferably 1:1.

7. The kit for treating onychomycoses or a bacterial nail infection according to claim 4, wherein the amino starter is 4-dimethylamino benzoic acid ethyl ester.
